(19) United States
(12) Patent Application Publication
(10) Pub. No.: US 2011/0251254 A1
(43) Pub. Date: Oct. 13, 2011

(54) NOVEL SALT OF TEGASEROD

(75) Inventors: Abhay Gaitonde, Maharashtra (IN); Bindu Manojkumar, Maharashtra (IN); Rahul Bhalerao, Maharashtra (IN); Sinderpal Tank, Maharashtra (IN)

(73) Assignees: Mylan India Private Limited, Maharashtra (IN); Generics [UK] Limited, Hertfordshire (GB)

(21) Appl. No.: 12/864,063

(22) PCT Filed: Nov. 27, 2008
(86) PCT No.: PCT/GB2008/051124

§ 371 Date: Jun. 1, 2011

(30) Foreign Application Priority Data
Jan. 23, 2008 (IN) 142KOL2008

(51) Int. Cl. A61K 31/4045; A61P 1/04; A61P 1/08; C07D 209/14

(52) U.S. Cl. 514/415; 548/507

(57) ABSTRACT

The present invention relates to a novel salt of tegaserod, namely tegaserod suberate, and to processes for the preparation thereof. The invention also relates to crystalline forms of the novel salt and to pharmaceutical compositions comprising the novel salt. Further, the invention relates to uses of said compositions in treating patients suffering from gastrointestinal disorders.

### CROSS-REFERENCE TO RELATED APPLICATION(S)

This application is a Section 371 National Stage Application of International No. PCT/GB2008/051124, filed 27 Nov. 2008 and published as WO 2009/092994 A1 on 30 Jul. 2009, which claims priority from the IN Patent Application No. 142/KOL/2008, filed 23 Jan. 2008, the contents of which are incorporated herein in their entirety for all purposes.

### FIELD OF THE INVENTION

The present invention relates to a novel salt of tegaserod, namely tegaserod suberate, and to processes for the preparation thereof. The invention also relates to crystalline forms of the novel salt and to pharmaceutical compositions comprising the novel salt. Further, the invention relates to uses of said compositions in treating patients suffering from gastrointestinal disorders.

### BACKGROUND OF THE INVENTION

Tegaserod, chemically named 2-[(5-methoxy-1H-indol-3-yl)methylene]-N-pentylhydrazine-carboximidamide, is a selective serotonin 4 (5-HT₄) receptor agonist, which can be used to treat gastrointestinal disorders such as heartburn, bloating, postoperative ileus, abdominal pain and discomfort, epigastric pain, nausea, vomiting, regurgitation, intestinal pseudo-obstruction, irritable bowel syndrome and gastro-oesophageal reflux. Tegaserod, as the maleate salt, is marketed for the short-term treatment of irritable bowel syndrome in women whose primary bowel symptom is constipation.

Tegaserod, represented by formula (I), was first described in U.S. Pat. No. 5,510,353. The maleate salt was also described, but a method of manufacturing tegaserod maleate is not disclosed. The only characterising data are the melting points which are disclosed as 190° C. for the maleate salt and 124° C. for the tegaserod base.

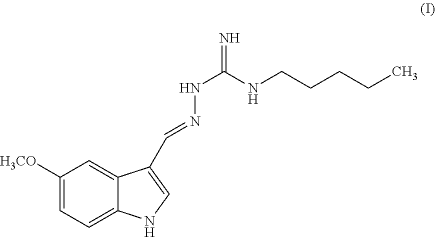

WO 2006/116953 describes crystalline forms of the hydrobromide, fumarate and oxalate salts of tegaserod. Also claimed is a process for preparing the hydrochloride, hydrobromide, fumarate, tartrate, citrate, lactate, mesylate, oxalate, succinate, glutarate, adipate, salicylate, sulphate, mandelate, camphor sulphonate and hydrogen sulphate salts of tegaserod from a specific crystalline form of tegaserod base. Another process described is a method of preparing the fumarate, maleate, tartrate, citrate, mesylate, lactate, succinate, oxalate, hydrochloride, salicylate, glutarate, adipate, hydrobromide, sulphate and hydrogen sulphate from a hydrogen halide salt of tegaserod.

There are often major hurdles to overcome before an active pharmaceutical ingredient (API) can be formulated into a composition that can be marketed. For example, the rate of dissolution of an API that has poor aqueous solubility is often problematic. The aqueous solubility is a major influence on the bioavailability of the API such that a poorly soluble API can mean the API is not available to have a pharmaceutical effect on the body. The API can also cause problems during manufacture of a pharmaceutical composition. For example, flowability, compactability and stickiness are all factors affected by the solid state properties of an API.

It has thus always been an aim of the pharmaceutical industry to provide many forms of an API in order to mitigate the problems described above. Different salts, crystalline forms also known as polymorphs, solvates, hydrates and amorphous forms are all forms of an API that can have different physiochemical and biological characteristics. Indeed, it has been discovered that the tegaserod maleate product on the market, Zelnorm®, has been linked to an increase in heart problems in a proportion of individuals. One possible reason is that the maleate moiety reacts with the tegaserod, resulting over time in the production of a toxic impurity. This impurity could be a contributor to the heart problems seen in some patients.

It would therefore be advantageous for the medicinal chemist to have a wide repertoire of alternative salts and crystalline forms of these and other known salts to aid in the preparation of products that are both efficacious and safe.

### SUMMARY OF THE INVENTION

Accordingly, the present invention provides a novel salt of tegaserod, namely tegaserod suberate, as well as a crystalline form of said salt.

As alluded to above, polymorphism influences every aspect of the solid state properties of an API and one of the important aspects of polymorphism in pharmaceuticals is the possibility of interconversion from one crystalline form to another. It is important that stable crystalline forms are used in pharmaceutical compositions as, for example, conversion from a form showing greater aqueous dissolution and potentially better bioavailability to a less soluble form can potentially have disastrous consequences.

Thus it is an object of the present invention to provide a novel tegaserod salt and a crystalline form according to the invention which may have an advantageous dissolution rate in vivo, leading to improved bioavailability, and further may provide advantageous characteristics during dosage form manufacture, for example, good conversion stability and formulation characteristics.

It is a further object of the present invention to provide a novel tegaserod salt and a crystalline form thereof which may have advantageous properties, for example, better solubility, bioavailability, stability including chemical and polymorphic stability, flowability, tractability, compressibility, compactability, toxicity, efficacy, or safety.

Accordingly, a first aspect according to the invention provides the compound tegaserod suberate or a tautomeric form thereof and/or a pharmaceutically acceptable solvate or hydrate thereof.

The tegaserod suberate may exist in one or more polymorphic, tautomeric, hydrate and/or solvate forms. The present invention embraces all polymorphic forms and their mixtures, all tautomeric forms and their mixtures, all hydrate forms and their mixtures, and all solvate forms and their mixtures. Although tegaserod is defined for convenience by reference to one guanidino form only, the invention is not to be understood as being in any way limited by the particular nomenclature or graphic representation employed.

In a second aspect according to the invention a novel crystalline form of tegaserod suberate is provided with a characteristic XRD spectrum having two or more peaks (preferably three or more, four or more, five or more, six or more, seven or more, eight or more, nine or more, ten or more, fifteen or more, or nineteen peaks) with 2θ values at 4.74, 8.11, 10.86, 11.60, 15.14, 15.61, 16.16, 17.06, 17.58, 18.51, 19.44, 20.52, 20.90, 21.80, 23.00, 24.28, 25.23, 26.24, 27.09±0.2° 2θ. In a particularly preferred embodiment tegaserod suberate is provided with a characteristic XRD spectrum having peaks with 2θ values at 4.74, 8.11, 10.86, 11.60, 15.14, 15.61, 16.16, 17.06, 17.58, 18.51, 19.44, 20.52, 20.90, 21.80, 23.00, 24.28, 25.23, 26.24, 27.09±0.2° 2θ. Preferably the tegaserod suberate has an XRPD trace substantially as shown in FIG. 1.

According to a third aspect of the present invention there is provided a crystalline form of tegaserod suberate characterised by a DSC with an endothermic peak at about 236.6° C.±0.5° C., preferably at about 236.60° C.±0.5° C. Preferably the tegaserod suberate has a DSC trace substantially as shown in FIG. 2.

The tegaserod suberate may have a TGA trace substantially as shown in FIG. 3.

Preferably the tegaserod suberate according to the above described aspects and embodiments has a chemical purity of greater than 95%, 96%, 97%, 98% or 99% (as measured by HPLC). Preferably the tegaserod suberate according to the above described aspects and embodiments has a polymorphic purity of greater than 95%, 96%, 97%, 98% or 99% (as measured by XRPD or DSC).

A fourth aspect provides a process for the preparation of tegaserod suberate according to the invention, preferably crystalline tegaserod suberate, comprising the steps of:

(a) mixing tegaserod and suberic acid in a solvent; and
(b) isolating the resultant salt.

In a preferred embodiment of the process the suberic acid is added as a solution of the free acid, preferably the solvent of the suberic acid solution is an aqueous solvent, most preferably the aqueous solvent is water. In a further embodiment, in step (b), the salt is isolated by filtration, preferably by vacuum filtration.

In a further embodiment, the tegaserod suberate is obtained on an industrial scale, preferably in batches of 0.5 kg, 1 kg, 5 kg, 10 kg, 50 kg, 100 kg, 500 kg or more.

A fifth aspect according to the invention provides a pharmaceutical composition comprising tegaserod suberate according to the invention or prepared according to the invention and one or more pharmaceutically acceptable excipients. Preferably the composition is a solid composition, most preferably a tablet or capsule composition.

A sixth aspect provides a composition according to the invention for use in the treatment or prevention of a gastrointestinal disorder, preferably selected from the list comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux. Preferably the gastrointestinal disorder is irritable bowel syndrome.

In a seventh aspect there is provided tegaserod suberate according to any of the aspects or embodiments described above for use as a medicament.

An eighth aspect provides tegaserod suberate according to the invention for use in the treatment or prevention of a gastrointestinal disorder. In a preferred embodiment the gastrointestinal disorder is selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux, most preferably the disorder is irritable bowel syndrome.

In a ninth aspect according to the invention there is further provided a method of treating or preventing a gastrointestinal disorder, preferably selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux (preferably irritable bowel syndrome), comprising administering to a patient in need thereof a composition comprising a pharmaceutically or prophylactically effective amount of tegaserod suberate according to any of the aspects or embodiments described above. Preferably the patient is a mammal, preferably a human.

A tenth aspect provides the use of tegaserod suberate according to any of the aspects or embodiments described above in the manufacture of a medicament for use in the treatment or prevention of a gastrointestinal disorder. In a preferred embodiment the gastrointestinal disorder is selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux, preferably irritable bowel syndrome.

### BRIEF DESCRIPTION OF THE ACCOMPANYING FIGURES

FIG. 1 shows the XRPD of tegaserod suberate.

FIG. 2 shows the DSC of tegaserod suberate.

FIG. 3 shows the TGA of tegaserod suberate.

### DETAILED DESCRIPTION OF THE INVENTION

As used herein the terms “crystalline form”, “polymorph” and “polymorphic form” are used interchangeably.

The terms “XRD” and “XRPD” are used interchangeably herein and preferably refer to an X-ray powder diffraction trace, spectrum or pattern.

The present invention provides the novel suberate salt of tegaserod and a process for its preparation. The process disclosed is simple and amenable to scale up and is capable of providing the salt in consistent crystalline and chemical purity of greater than 95% respectively, preferably greater than 96%, more preferably greater than 97%. Particularly preferred is a purity of greater than 98% and most preferred is a purity of greater than 99% irrespective of the scale of preparation.

A preferred process according to the invention for preparing tegaserod suberate according to the invention comprises mixing tegaserod and suberic acid. Preferably the suberic acid is dissolved in a solvent. In a particularly preferred embodiment the solvent is an aqueous solvent, most preferably water. Preferably the tegaserod is in the form of the free base. Of course it will be understood that the tegaserod can be completely or only partially dissolved in one or a mixture of solvent(s) and the process still falls within the scope of the invention, and further that the tegaserod, suberic acid and solvent can be combined in any order and the process remains within the scope of the invention. For example certain embodiments according to the invention comprise adding tegaserod to the solvent(s), to which is added the suberic acid, preferably in solution. Whatever their nature the suberic acid solution and the tegaserod solution should be miscible to create a single phase. Preferably when the suberic acid is dissolved in an aqueous solvent, the solvent in which the tegaserod is dissolved should be miscible with the aqueous suberic acid solution. The inventors have found that C₁-C₆ alcohols are advantageous, preferably selected from the group comprising methanol, ethanol, isopropyl alcohol (IPA), tert-butanol and isobutanol, more preferably methanol, but other polar organic solvents, especially polar protic organic solvents, capable of dissolving tegaserod can be utilised.

The resulting reaction mixture comprising the tegaserod and the suberic acid, in certain embodiments can be stirred to increase the precipitation of the solid salt. It is preferred that the stirring occurs at between about 20-30° C. or approximately room temperature, but it is envisaged that the stirring conditions may be varied and still remain within the scope of the invention. Preferably the reaction mixture comprising the tegaserod and the suberic acid is stirred for between about 0.5-3 hours.

The solid product obtained can then be isolated by any means common in the field or known to the skilled artisan. In one embodiment the solid salt is obtained by evaporation of the solvent. However, in a particularly preferred embodiment the solid product is filtered. Preferably the product is dried at a temperature that does not induce conversion of the crystalline form obtained or cause the salt or crystalline form to degrade. The inventors have found that drying the product at about 35-40° C. is advantageous. Preferably, in certain embodiments the solid product is dried under vacuum until a constant weight is obtained.

The tegaserod suberate according to the invention may also be further purified if required. The inventors have found that dissolving the tegaserod suberate in an organic solvent and then causing the salt to precipitate from the solution results in particularly pure tegaserod suberate. In preferred embodiments the organic solvent is a protic or aprotic solvent, preferably tetrahydrofuran (THF). Preferably, the mixture is heated to facilitate dissolution of the tegaserod suberate, in certain embodiments to between about 40-90° C., most preferably to between about 60-80° C., when the solvent is THF. The mixture may then be cooled to precipitate the tegaserod suberate or in alternative embodiments an anti-solvent may be added. In preferred embodiments the mixture is cooled to between about 20-30° C. The resultant precipitated solid can be isolated by any means, preferably by vacuum filtration. The solid obtained may then be washed, preferably with THF, and is preferably dried, preferably at about 40° C., preferably until a constant weight is achieved.

The inventors have found that preparation of tegaserod suberate as described above and further crystallisation from solvents such as THF result in tegaserod suberate having both chemical and crystalline purity of greater than 99%.

Illustrative of the invention is a pharmaceutical composition comprising tegaserod suberate and one or more pharmaceutical excipients. In a further aspect a process for preparing the composition is provided comprising mixing tegaserod suberate according to the invention and a pharmaceutically acceptable excipient. A yet further aspect of the invention provides treatment of a 5-HT₄ receptor mediated disorder in a subject in need thereof comprising administering to the subject a composition comprising a therapeutically effective amount of tegaserod suberate. 5-HT₄ receptor mediated disorders comprise gastrointestinal disorders such as heartburn, bloating, postoperative ileus, abdominal pain and discomfort, epigastric pain, nausea, vomiting, regurgitation, intestinal pseudo-obstruction, irritable bowel syndrome and gastro-oesophageal reflux.

In another aspect according to the invention there is provided tegaserod suberate for treating a 5-HT₄ receptor mediated disorder in a subject in need thereof. Of course, it will be realised that the tegaserod suberate may be in amorphous form or any of a number of crystalline forms.

In addition to the active ingredient(s), the pharmaceutical compositions of the present invention may contain one or more excipients. Excipients are added to the composition for a variety of purposes. Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulphate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. Carbopol®), carboxymethyl cellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminium silicate, maltodextrin, methyl cellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethyl cellulose calcium, carboxymethyl cellulose sodium (e.g. Ac-Di-Sol®, Primellose®), colloidal silicon dioxide, croscarmellose sodium, crospovidone (e.g. Kollidon®, Polyplasdone®), guar gum, magnesium aluminium silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulphate, sodium stearyl fumarate, stearic acid, talc and zinc stearate.

Flavouring agents and flavour enhancers make the dosage form more palatable to the patient. Common flavouring agents and flavour enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the tegaserod salt and any other solid excipients are dissolved or suspended in a liquid carrier such as water, vegetable oil, alcohol, polyethylene glycol, propylene glycol or glycerine.

Liquid pharmaceutical compositions may further contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel or organoleptic qualities of the product and/or coat the lining of the gastrointestinal tract. Such agents include acacia, alginic acid, bentonite, carbomer, carboxymethyl cellulose calcium or sodium, cetostearyl alcohol, methyl cellulose, ethyl cellulose, gelatin, guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxytoluene, butylated hydroxyanisole and ethylenediaminetetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts. Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or a soft shell. The shell may be made from gelatin and optionally contain a plasticizer such as glycerine and sorbitol, and an opacifying agent or colourant. The active ingredient and excipients may be formulated into compositions and dosage forms according to methods known in the art.

A composition for tableting or capsule filling may be prepared by wet granulation. In wet granulation, some or all of the active ingredient and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry granulation. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

In further embodiments the composition of the invention may further comprise one or more additional active ingredients. Further active ingredients may include other 5-HT₄ receptor agonists such as prucalopride, RS 67333 (1-(4-amino-5-chloro-2-methoxyphenyl)-3-(1-n-butyl-4-piperidinyl)-1-propanone), RS 67506 (1-(4-amino-5-chloro-2-methoxyphenyl)-3-[1-[2-[(methylsulphonyl)amino]ethyl]-4-piperidinyl]-1-propanone), cisapride, renzapride, norcisapride, mosapride, zacopride, SB 205149, SC 53116, BIMU 1, and BIMU 8; proton pump inhibitors such as omeprazole, rabeprazole, pantoprazole, and lansoprazole; 5-HT₃ receptor agonists such as cilansetron which is described in EP 297 651, alosetron which is described in WO 99/17755, ramosetron, azasetron, ondansetron, dolasetron, granisetron, and tropisetron; selective serotonin reuptake inhibitors such as citalopram, escitalopram, fluoxetine, fluvoxamine, sertraline, paroxetine, zimeldine, norzimeldine, clomipramine, alaproclate, venlafaxine, cericlamine, duloxetine, milnacipran, nefazodone, OPC 14503, and cyanodothiepin; and dipeptidyl peptidase IV (DPP-IV) inhibitors. Of course it will be obvious that the above is not an exhaustive list.

The following paragraphs enumerated consecutively from 1 through 30 provide for various aspects of the present invention. In one embodiment, the present invention provides:

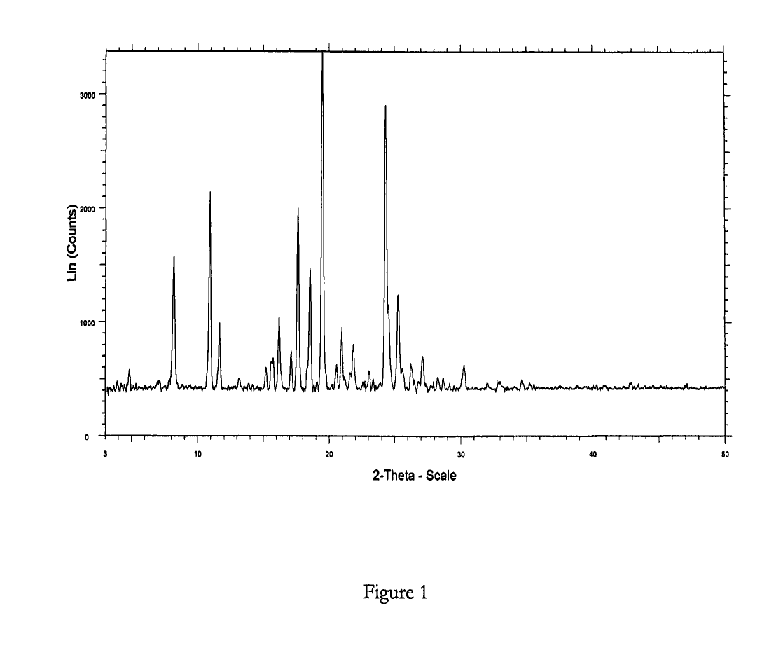

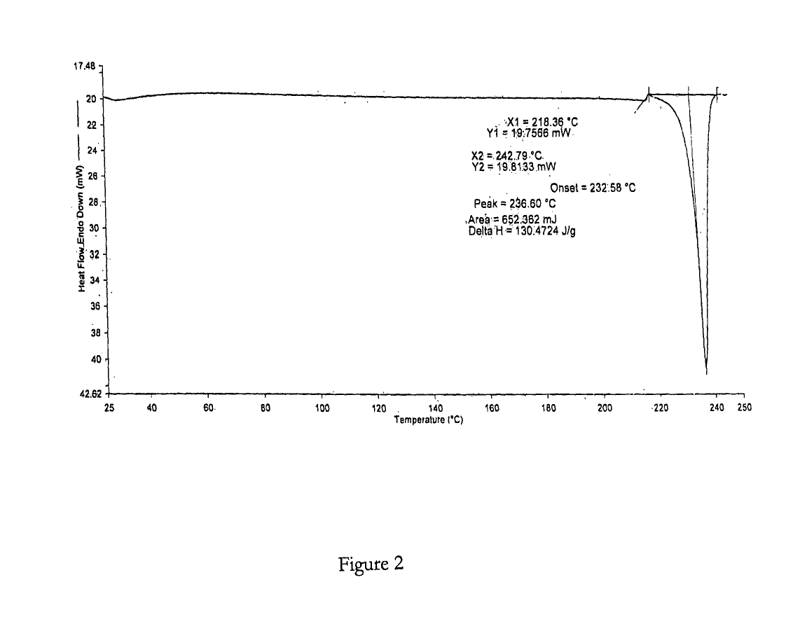

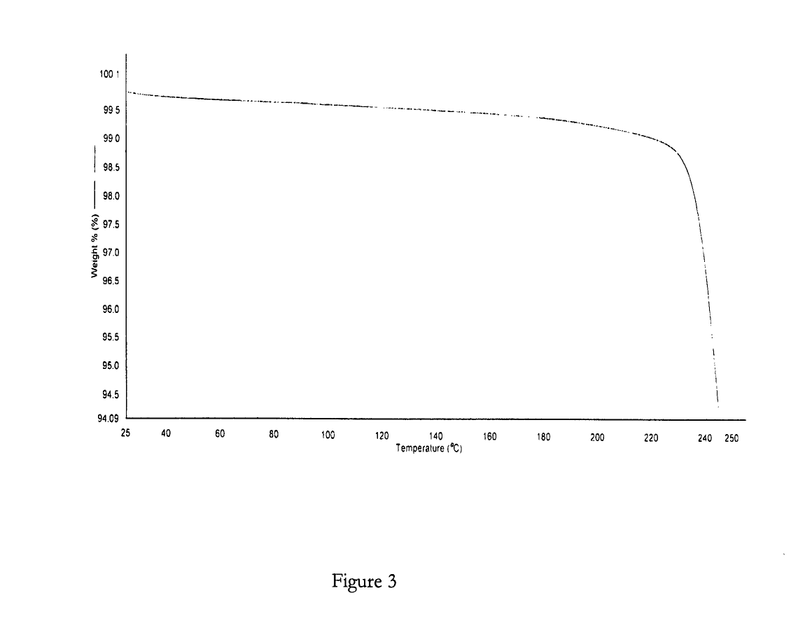

1. Tegaserod suberate or a tautomeric form thereof and/or a pharmaceutically acceptable solvate or hydrate thereof.
2. A crystalline form of tegaserod suberate with a characteristic XRD spectrum having two or more peaks with 2θ values at 4.74, 8.11, 10.86, 11.60, 15.14, 15.61, 16.16, 17.06, 17.58, 18.51, 19.44, 20.52, 20.90, 21.80, 23.00, 24.28, 25.23, 26.24, 27.09±0.2° 2θ.
3. A crystalline form of tegaserod suberate having an XRPD trace substantially as shown in FIG. 1.
4. A crystalline form of tegaserod suberate characterised by a DSC with an endothermic peak at about 236.6° C.±0.5° C.
5. A crystalline form of tegaserod suberate having a DSC trace substantially as shown in FIG. 2.
6. A crystalline form of tegaserod suberate having a TGA trace substantially as shown in FIG. 3.
7. Tegaserod suberate according to any one of paragraphs 1-6, having a chemical purity of greater than 95% (as measured by HPLC).
8. Tegaserod suberate according to any one of paragraphs 1-7, having a polymorphic purity of greater than 95% (as measured by XRPD or DSC).
9. A process for the preparation of tegaserod suberate according to any one of paragraphs 1-8, comprising the steps of:
(a) mixing tegaserod and suberic acid in a solvent; and
(b) isolating the resultant salt.
10. A process according to paragraph 9, wherein the suberic acid is added as a solution of the free acid.
11. A process according to paragraph 10, wherein the solution comprises an aqueous solvent and suberic acid.
12. A process according to paragraph 11, wherein the aqueous solvent is water.
13. A pharmaceutical composition comprising tegaserod suberate according to any one of paragraphs 1-8 or prepared by a process according to any one of paragraphs 9-12, and one or more pharmaceutically acceptable excipients.
14. A composition according to paragraph 13, wherein the composition is a solid composition.
15. A composition according to paragraph 14, wherein the composition is a tablet or capsule composition.
16. A composition according to any one of paragraphs 13-15, for use in the treatment or prevention of a gastrointestinal disorder.
17. A composition according to paragraph 16, wherein the gastrointestinal disorder is selected from the list comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux.
18. A composition according to paragraph 17, wherein the gastrointestinal disorder is irritable bowel syndrome.
19. Tegaserod suberate according to any one of paragraphs 1-8 or prepared by a process according to any one of paragraphs 9-12, for use as a medicament.
20. Tegaserod suberate according to paragraph 19, for use in the treatment or prevention of a gastrointestinal disorder.
21. Tegaserod suberate according to paragraph 20, wherein the gastrointestinal disorder is selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux.
22. Tegaserod suberate according to paragraph 21, wherein the gastrointestinal disorder is irritable bowel syndrome.
23. A method of treating or preventing a gastrointestinal disorder, comprising administering to a patient in need thereof a therapeutically of prophylactically effective amount of tegaserod suberate according to any one of paragraphs 1-8 or 19-22, or of tegaserod suberate prepared by a process according to any one of paragraphs 9-12, or of a pharmaceutical composition according to any one of paragraphs 13-18.
24. A method according to paragraph 23, wherein the gastrointestinal disorder is selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux.
25. A method according to paragraph 24, wherein the gastrointestinal disorder is irritable bowel syndrome.
26. A method according to any one of paragraphs 23-25, wherein the patient is a mammal.
27. A method according to paragraph 26, wherein the mammal is a human.
28. Use of tegaserod suberate according to any one of paragraphs 1-8 or 19-22, or prepared by a process according to any one of paragraphs 9-12, in the manufacture of a medicament for use in the treatment or prevention of a gastrointestinal disorder.
29. Use according to paragraph 28, wherein the gastrointestinal disorder is heartburn, bloating, postoperative ileus, abdominal pain or discomfort, epigastric pain, nausea, vomiting, regurgitation, intestinal pseudo-obstruction, irritable bowel syndrome or gastro-oesophageal reflux.
30. Use according to paragraph 29, wherein the gastrointestinal disorder is irritable bowel syndrome.

The details of the invention, its objects and advantages are explained hereunder in greater detail in relation to non-limiting exemplary illustrations.

### EXAMPLES

### Example 1

### Preparation of Tegaserod Suberate

Tegaserod (1 eq) was taken in 3.3 volumes of methanol at 25-30° C. To this slurry was added a solution of suberic acid (2 eq) in 5 volumes of water and the mixture was stirred for about 30 minutes at 25-30° C. The precipitated salt was filtered and washed with 5 volumes of water and dried at 35° C. under vacuum for about 2 hours.

¹H-NMR indicated formation of tegaserod suberate.

Yield=78%

Chemical purity>99% (as measured by HPLC)

Polymorphic purity>99% (as measured by DSC)

### Example 2

### Purification of Crude Tegaserod Suberate

1 g of tegaserod suberate was added to 40 volumes of THF and heated to about 66° C. for about 10 minutes. The reaction mixture was then cooled to between about 25-30° C. for about 30 minutes. The slurry was filtered and the solid product was dried at 35° C. under vacuum until a constant weight was obtained.

¹H-NMR indicated formation of tegaserod suberate. XRPD data confirmed that the tegaserod suberate product obtained had a crystalline structure.

Yield=99%

Chemical purity>99% (as measured by HPLC)

Polymorphic purity>99% (as measured by DSC)

1-30. (canceled)

31. Tegaserod suberate or a tautomeric form thereof and/or a pharmaceutically acceptable solvate or hydrate thereof.

32. Tegaserod suberate according to claim 31, having:
(i) a chemical purity of greater than 95% (as measured by HPLC); and/or
(ii) a polymorphic purity of greater than 95% (as measured by XRPD or DSC).

33. A crystalline form of tegaserod suberate or a tautomeric form thereof and/or a pharmaceutically acceptable solvate or hydrate thereof.

34. The crystalline form according to claim 33:
(i) with a characteristic XRD spectrum having two or more peaks with 2θ values at 4.74, 8.11, 10.86, 11.60, 15.14, 15.61, 16.16, 17.06, 17.58, 18.51, 19.44, 20.52, 20.90, 21.80, 23.00, 24.28, 25.23, 26.24, 27.09±0.2° 2θ; and/or
(ii) having an XRPD trace substantially as shown in FIG. 1; and/or
(iii) characterised by a DSC with an endothermic peak at about 236.6° C.±0.5° C.; and/or
(iv) having a DSC trace substantially as shown in FIG. 2; and/or
(v) having a TGA trace substantially as shown in FIG. 3.

35. The crystalline form according to claim 33, having:
(i) a chemical purity of greater than 95% (as measured by HPLC); and/or
(ii) a polymorphic purity of greater than 95% (as measured by XRPD or DSC).

36. A process for the preparation of tegaserod suberate according to claim 31, comprising the steps of:
(a) mixing tegaserod and suberic acid in a solvent; and
(b) isolating the resultant salt.

37. A process according to claim 36, wherein the suberic acid is added as:
(i) a solution of the free acid; and/or
(ii) a solution comprising an aqueous solvent and suberic acid; and/or
(iii) a solution comprising water and suberic acid.

38. A process for the preparation of the crystalline form according to claim 33, comprising the steps of:
(a) mixing tegaserod and suberic acid in a solvent; and
(b) isolating the resultant salt.

39. A process according to claim 38, wherein the suberic acid is added as:
(i) a solution of the free acid; and/or
(ii) a solution comprising an aqueous solvent and suberic acid; and/or
(iii) a solution comprising water and suberic acid.

40. A pharmaceutical composition comprising tegaserod suberate according to claim 31, and one or more pharmaceutically acceptable excipients.

41. A composition according to claim 40, wherein the composition is:
(i) a solid composition; and/or
(ii) a tablet or capsule composition; and/or
(iii) for use in the treatment or prevention of a gastrointestinal disorder; and/or
(iv) for use in the treatment or prevention of a gastrointestinal disorder selected from the list comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux; and/or
(v) for use in the treatment or prevention of irritable bowel syndrome.

42. A pharmaceutical composition comprising the crystalline form according to claim 33, and one or more pharmaceutically acceptable excipients.

43. A composition according to claim 42, wherein the composition is:
(i) a solid composition; and/or
(ii) a tablet or capsule composition; and/or
(iii) for use in the treatment or prevention of a gastrointestinal disorder; and/or
(iv) for use in the treatment or prevention of a gastrointestinal disorder selected from the list comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux; and/or
(v) for use in the treatment or prevention of irritable bowel syndrome.

44. Tegaserod suberate according to claim 31, for use:
(i) as a medicament; and/or
(ii) in the treatment or prevention of a gastrointestinal disorder; and/or
(iii) in the treatment or prevention of a gastrointestinal disorder selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux; and/or
(iv) in the treatment or prevention of irritable bowel syndrome.

45. The crystalline form according to claim 33, for use:
(i) as a medicament; and/or
(ii) in the treatment or prevention of a gastrointestinal disorder; and/or
(iii) in the treatment or prevention of a gastrointestinal disorder selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux; and/or
(iv) in the treatment or prevention of irritable bowel syndrome.

46. A method of treating or preventing a gastrointestinal disorder, comprising administering to a patient in need thereof a therapeutically of prophylactically effective amount of tegaserod suberate according to claim 31.

47. A method according to claim 46, wherein the gastrointestinal disorder is:
(i) selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux; and/or
(ii) irritable bowel syndrome.

48. A method according to claim 46, wherein the patient is:
(i) a mammal; and/or
(ii) a human.

49. A method of treating or preventing a gastrointestinal disorder, comprising administering to a patient in need thereof a therapeutically of prophylactically effective amount of the crystalline form according to claim 33.

50. A method according to claim 49, wherein the gastrointestinal disorder is:
(i) selected from the group comprising heartburn, bloating, postoperative ileus, abdominal pain and discomfort, epigastric pain, nausea, vomiting, regurgitation, intestinal pseudo-obstruction, irritable bowel syndrome and gastro-oesophageal reflux; and/or
(ii) irritable bowel syndrome.

51. A method according to claim 49, wherein the patient is:
(i) a mammal; and/or
(ii) a human.
